(19) United States
(12) Patent Application Publication
(10) Pub. No.: US 2024/0225582 A1
(43) Pub. Date: Jul. 11, 2024

(54) MEDICAL IMAGE PROCESSING APPARATUS, MEDICAL IMAGE PROCESSING METHOD, AND STORAGE MEDIUM

(71) Applicant: CANON MEDICAL SYSTEMS CORPORATION, Otawara-shi (JP)

(72) Inventor: Keitaro KAWASHIMA, Nasushiobara (JP)

(73) Assignee: CANON MEDICAL SYSTEMS CORPORATION, Otawara-shi (JP)

(21) Appl. No.: 18/409,058

(22) Filed: Jan. 10, 2024

(30) Foreign Application Priority Data
Jan. 11, 2023 (JP) 2023-002635

(51) Int. Cl. A61B 6/00; A61B 6/50; G06T 7/00; G16H 50/50

(52) U.S. Cl. CPC A61B 6/5217; A61B 6/503; G06T 7/0012; G16H 50/50; G06T 2200/04; G06T 2207/10081; G06T 2207/30048

(57) ABSTRACT

A medical image processing apparatus according to an embodiment includes processing circuitry. The processing circuitry is configured to obtain three-dimensional medical image data; to specify, on the basis of a left atrium region, a left ventricle region, and an aortic valve region rendered in the three-dimensional medical image data, a three-chamber long-axis cross-sectional plane; to specify a mitral valve axis on the basis of a first annulus region in the three-dimensional medical image data and the three-chamber long-axis cross-sectional plane; to specify, from the three-chamber long-axis cross-sectional plane, an anterior leaflet region and a second annulus region; to specify an aortic valve axis on the basis of a positional relationship between the anterior leaflet region and the second annulus region on the three-chamber long-axis cross-sectional plane; and to calculate an angle based on the mitral valve axis and the aortic valve axis.

### CROSS-REFERENCE TO RELATED APPLICATIONS

This application is based upon and claims the benefit of priority from Japanese Patent Application No. 2023-002635, filed on Jan. 11, 2023, the entire contents of which are incorporated herein by reference.

### FIELD

Embodiments described herein relate generally to a medical image processing apparatus, a medical image processing method, and a storage medium.

### BACKGROUND

Conventionally, various types of indices for understanding hemodynamics of the heart are measured by using a medical image obtained by a medical image diagnosis apparatus such as an X-ray Computed Tomography (CT) apparatus. For example, as an index indicating a stenosis state of the Left Ventricular Outflow Tract (which hereinafter may be referred to as “LVOT”), which is a blood flow path from the left ventricle to the aortic valves, an Aorto-Mitral Angle (AMA) is measured to obtain the angle formed by a line (which may be referred to as a “mitral valve axis”) perpendicular to a straight line connecting together points on the mitral valve annulus and another line (which may be referred to as an “aortic valve axis”) perpendicular to a straight line connecting together points on the aortic valve annulus on a three-chamber long-axis cross-sectional plane.

### BRIEF DESCRIPTION OF THE DRAWINGS

FIG. 1 is a block diagram illustrating an exemplary configuration of a medical image processing system according to an embodiment;

FIG. 2 is a block diagram illustrating an exemplary configuration of an X-ray CT apparatus according to the embodiment;

FIG. 3 is a drawing for explaining an example of a process of specifying a three-chamber long-axis cross-sectional plane according to the embodiment;

FIG. 4 is a drawing for explaining an example of a process of specifying a mitral valve axis according to the embodiment;

FIG. 5 is a drawing for explaining an example of a process of specifying an aortic valve axis according to the embodiment;

FIG. 6 is a drawing for explaining an example of a process of calculating an AMA according to the embodiment;

FIG. 7 is a flowchart illustrating an example of processes performed by a medical image processing apparatus according to the embodiment;

FIG. 8 is a drawing for explaining an example of an AMA calculation result obtained by the medical image processing apparatus according to the embodiment;

FIG. 9 is a drawing for explaining an example of another AMA calculation result from processes different from those in the embodiment; and

FIG. 10 is a drawing for explaining an example of yet another AMA calculation result obtained by a medical image processing apparatus different from that in the embodiment.

### DETAILED DESCRIPTION

A medical image processing apparatus according to an embodiment includes processing circuitry. The processing circuitry is configured to obtain three-dimensional medical image data rendering the heart; to specify, on the basis of a left atrium region, a left ventricle region, and an aortic valve region rendered in the three-dimensional medical image data, a three-chamber long-axis cross-sectional plane that traverses across these regions; to specify a mitral valve axis on the basis of a first annulus region included in a mitral valve region rendered in the three-dimensional medical image data and the three-chamber long-axis cross-sectional plane; to specify, from the three-chamber long-axis cross-sectional plane, an anterior leaflet region included in the mitral valve region and a second annulus region included in the aortic valve region; to specify an aortic valve axis on the basis of a positional relationship between the anterior leaflet region and the second annulus region on the three-chamber long-axis cross-sectional plane; and to calculate an angle based on the mitral valve axis and the aortic valve axis.

Exemplary embodiments of a medical image processing apparatus, a medical image processing method, and a storage medium will be explained in detail below, with reference to the accompanying drawings. In the embodiments described below, an example of a medical image processing system will be explained, which includes an X-ray CT apparatus serving as an example of a medical image diagnosis apparatus and the medical image processing apparatus. Further, the explanation will be based on the assumption that the processes described below are performed on the basis of projection data acquired by the X-ray CT apparatus.

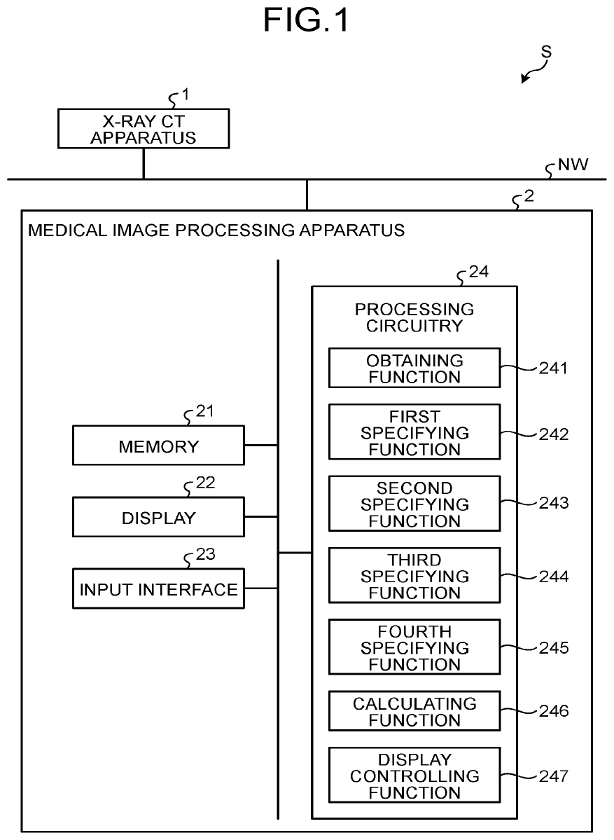

FIG. 1 is a block diagram illustrating an exemplary configuration of a medical image processing system S according to an embodiment. An X-ray CT apparatus 1 and a medical image processing apparatus 2 are connected to each other via a network NW.

As long as the two apparatuses are connectable via the network NW, installation locations of the X-ray CT apparatus 1 and the medical image processing apparatus 2 are arbitrary. For example, the X-ray CT apparatus 1 and the medical image processing apparatus 2 maybe installed in mutually the same facility such as a hospital or may be installed in mutually-different facilities. In other words, the network NW may be configured as a local network that is closed in a facility or may be a network intermediated by the Internet.

Further, communication between the X-ray CT apparatus 1 and the medical image processing apparatus 2 may be performed via another apparatus such as an image storage apparatus or may be performed directly without being intermediated by other apparatuses. Examples of the image storage apparatus include a server of a Picture Archiving and Communication System (PACS).

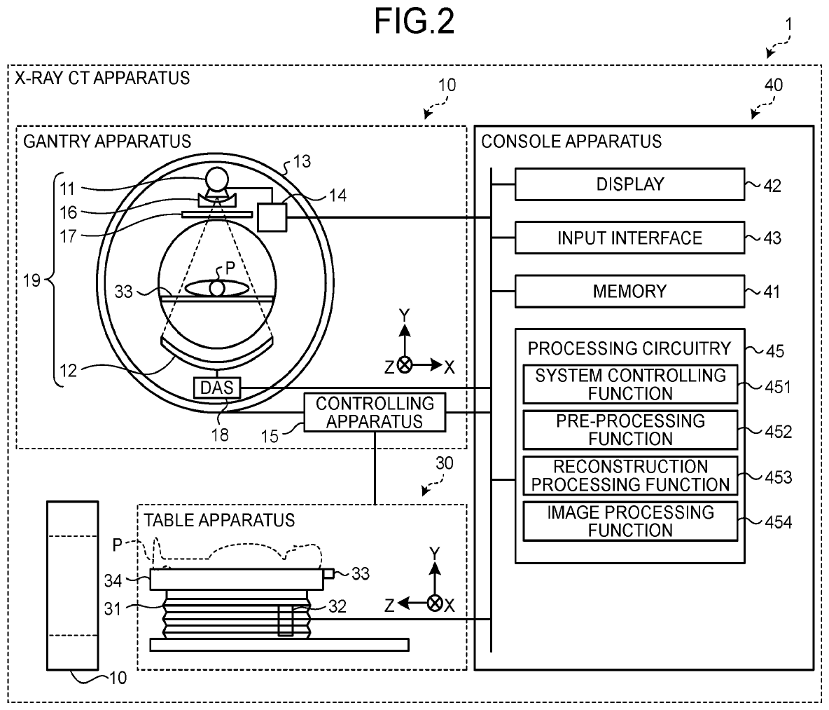

To begin with, the X-ray CT apparatus 1 will be explained, with reference to FIG. 2. FIG. 2 is a block diagram illustrating an exemplary configuration of the X-ray CT apparatus 1 according to the embodiment. As illustrated in FIG. 2, the X-ray CT apparatus 1 includes a gantry apparatus 10, a table apparatus 30, and a console apparatus 40.

In the present embodiment, the longitudinal direction of a rotation axis of a rotating frame 13 in a non-tilt state is defined as a Z axis; the direction orthogonal to the Z axis and extending from a rotation center to a post supporting the rotating frame 13 is defined as an X axis; and the direction orthogonal to the Z axis and the X axis is defined as a Y axis.

The gantry apparatus 10 includes an imaging system 19 for imaging medical images to be used for diagnosing purposes. For example, the imaging system 19 is configured with an X-ray tube 11, a X-ray detector 12, a wedge 16, and a collimator 17. In other words, the gantry apparatus 10 is an apparatus including the imaging system 19 configured to radiate X-rays onto an examined subject (hereinafter “patient”) P and to acquire projection data from detection data of X-rays that have passed through the patient P.

Further, the gantry apparatus 10 has an opening part used for housing the patient P therein. A tabletop 33 on which the patient P is placed is stored into the opening part while the side having the table apparatus 30 is used as an entrance.

The gantry apparatus 10 includes the X-ray tube 11, the wedge 16, the collimator 17, the X-ray detector 12, an X-ray high-voltage apparatus 14, a Data Acquisition System (DAS) 18, the rotating frame 13, a controlling apparatus 15, and the table apparatus 30.

The X-ray tube 11 is a vacuum tube in which thermo electrons are emitted from a negative pole (a filament) toward a positive pole (a target or an anode), with application of high voltage from the X-ray high-voltage apparatus 14. For instance, examples of the X-ray tube 11 include a rotating anode X-ray tube configured to generate X-rays by having the thermo electrons emitted onto a rotating anode.

The wedge 16 is a filter for adjusting the X-ray amount of the X-rays emitted from the X-ray tube 11. More specifically, the wedge 16 is a filter configured to pass and attenuate the X-rays emitted from the X-ray tube 11 so that the X-rays emitted from the X-ray tube 11 onto the patient P has a predetermined distribution.

For example, the wedge 16 maybe a wedge filter or a bow-tie filter and is a filter obtained by processing aluminum so as to have a predetermined target angle and a predetermined thickness.

The collimator 17 is realized with lead plates or the like for narrowing down an emission range of the X-rays that have passed through the wedge 16 and is configured to form a slit with a combination of the plurality of lead plates or the like. The collimator 17 maybe referred to as an X-ray limiter in some situations.

The X-ray detector 12 is configured to detect the X-rays that were emitted from the X-ray tube 11 and have passed through the patient P and is configured to output an electrical signal corresponding to the amount of the X-rays to a data acquisition apparatus (the DAS 18). For example, the X-ray detector 12 includes a plurality of columns of X-ray detecting elements in each of which a plurality of X-ray detecting elements are arranged in a channel direction along an arc while being centered on a focal point of the X-ray tube 11. In this situation, the channel direction denotes the circumferential direction of the rotating frame 13.

For example, the X-ray detector 12 includes the plurality of columns of X-ray detecting elements in each of which the plurality of X-ray detecting elements are arranged in the channel direction along an arc while being centered on the focal point of the X-ray tube 11. For example, the X-ray detector 12 has a structure in which the plurality of columns of X-ray detecting elements are arranged in a slice direction (which may be called a body-axis direction or a column direction), while each of the columns includes the plurality of X-ray detecting elements arranged in the channel direction.

Further, for example, the X-ray detector 12 is an indirect-conversion type detector including a grid, a scintillator array, and an optical sensor array. The scintillator array includes a plurality of scintillators. Each of the scintillators includes a scintillator crystal that outputs light in a photon quantity corresponding to the amount of incident X-rays. The grid is arranged on a surface of the scintillator array that is positioned on the X-ray incident side and includes an X-ray blocking plate having a function of absorbing scattered X-rays.

The optical sensor array has a function of converting light into electrical signals in correspondence with the amounts of light from the scintillators and includes, for example, optical sensors such as Photomultiplier Tubes (PMTs). Alternatively, the X-ray detector 12 maybe a direct-conversion type detector including a semiconductor element configured to convert incident X-rays into electrical signals.

The X-ray high-voltage apparatus 14 includes a high-voltage generating apparatus including electrical circuitry such as a transformer, a rectifier, and the like and having a function of generating the high voltage to be applied to the X-ray tube 11; and an X-ray controlling apparatus configured to control output voltage corresponding to the X-rays to be emitted by the X-ray tube 11. The high-voltage generating apparatus may be of a transformer type or an inverter type.

Further, the X-ray high-voltage apparatus 14 maybe provided for the rotating frame 13 or may be provided so as to belong to a fixed frame (not illustrated) of the gantry apparatus 10. In this situation, the fixed frame denotes a frame configured to rotatably support the rotating frame 13.

The DAS 18 includes an amplifier configured to perform an amplifying process on the electrical signals output from the X-ray detecting elements included in the X-ray detector 12 and an Analog/Digital (A/D) converter configured to convert electrical signals into digital signals. The DAS 18 is configured to generate the detection data. The detection data generated by the DAS 18 is transferred to the console apparatus 40. The detection data may be sinogram, for example.

The sinogram is data presenting projection data generated in correspondence with each position (hereinafter, “view angle”) of the X-ray tube 11 and in correspondence with each of the X-ray detecting elements, so as to be kept in correspondence with a view direction and the channel direction. In this situation, the view direction corresponds to the view angle and denotes the direction of the X-ray emission.

In this situation, when a single scan is performed by using only one of the detecting element columns in the X-ray detector 12, it is possible to generate one sinogram with respect to the one scan. In contrast, when a helical scan or a volume scan is performed by using two or more of the detecting element columns in the X-ray detector 12, it is possible to generate a plurality of sinograms with respect to the one scan.

The rotating frame 13 is an annular frame configured to support the X-ray tube 11 and the X-ray detector 12 so as to oppose each other and configured to rotate the X-ray tube 11 and the X-ray detector 12 via the controlling apparatus 15. In this situation, in addition to the X-ray tube 11 and the X-ray detector 12, the rotating frame 13 is configured to further support the X-ray high-voltage apparatus 14 and the DAS 18.

The rotating frame 13 is rotatably supported by a non-rotating part (e.g., the fixed frame; not illustrated in FIG. 2) of the gantry apparatus. A rotating mechanism includes, for example, a motor configured to generate a rotation drive force and a bearing configured to rotate the rotating frame 13 by transmitting the rotation drive force thereto. The motor is provided in the non-rotating part, for example. The bearing is physically connected to the rotating frame 13 and the motor, so that the rotating frame 13 rotates in accordance with rotating force of the motor.

The rotating frame 13 and the non-rotating part are each provided with communication circuitry that uses a contactless or contact method. With this arrangement, a unit supported by the rotating frame 13 is configured to communicate with the non-rotating part or an external apparatus of the gantry apparatus 10.

When optical communication is adopted as the contactless communication method, for example, the detection data generated by the DAS 18 is transmitted through optical communication from a transmitter including a light emitting diode (LED) and being provided on the rotating frame 13, to a receiver including a photodiode and being provided in a non-rotating part of the gantry apparatus, and is further transferred by the transmitter from the non-rotating part to the console apparatus 40.

Besides the above, as the communication method, it is also acceptable to alternatively adopt a contactless data transfer method based on a capacity coupling scheme or a radio wave scheme, as well as a contact data transfer method using a slip ring and an electrode brush.

The controlling apparatus 15 includes processing circuitry having a Central Processing Unit (CPU) or the like and a driving mechanism such as a motor and an actuator or the like. The controlling apparatus 15 has a function of receiving input signals from an input interface 43 (explained later) attached to the console apparatus 40 or to the gantry apparatus 10 and controlling operations of the gantry apparatus 10 and the table apparatus 30.

For example, upon receipt of the input signals, the controlling apparatus 15 is configured to exercise control to rotate the rotating frame 13, control to tilt the gantry apparatus 10, and control to bring the table apparatus 30 and the tabletop 33 into operation. In this situation, the control to tilt the gantry apparatus 10 is realized as a result of the controlling apparatus 15 rotating the rotating frame 13 on an axis parallel to the X-axis direction, according to an inclination angle (a tilt angle) input through an input interface attached to the gantry apparatus 10.

Further, the controlling apparatus 15 may be provided for the gantry apparatus 10 or may be provided for the console apparatus 40.

The table apparatus 30 is an apparatus on which the patient P to be scanned is placed and moved and includes a base 31, a table driving apparatus 32, the tabletop 33, and a supporting frame 34. The base 31 is a casing configured to support the supporting frame 34 so as to be movable in vertical directions. The table driving apparatus 32 is a motor or an actuator configured to move the tabletop 33 on which the patient P is placed in the longitudinal directions (the Z-axis directions in FIG. 2) of the tabletop 33.

The tabletop 33 provided on the top face of the supporting frame 34 is a board on which the patient P is placed. Further, in addition to the tabletop 33, the table driving apparatus 32 maybe configured to move the supporting frame 34 in the longitudinal directions of the tabletop 33.

The table driving apparatus 32 is configured to move the base 31 in up-and down directions according to control signals from the controlling apparatus 15. Further, the table driving apparatus 32 is configured to move the tabletop 33 in the longitudinal directions (the Z-axis directions) according to control signals from the controlling apparatus 15.

The console apparatus 40 is an apparatus configured to receive operations performed by an operator on the X-ray CT apparatus 1 and to reconstruct X-ray CT image data from the X-ray detection data acquired by the gantry apparatus 10. The console apparatus 40 includes a memory 41, a display 42, the input interface 43, and processing circuitry 45.

For example, the memory 41 is realized by using a semiconductor memory element such as a Random Access Memory (RAM) or a flash memory, or a hard disk, an optical disc, or the like. The memory 41 is configured to store therein the projection data and reconstructed image data, for example. Further, the memory 41 is configured to store therein imaging protocols.

In this situation, an imaging protocol defines a procedure or the like for obtaining an image by controlling the imaging system 19 and imaging the patient P. For example, the imaging protocol is represented by a parameter group related to an imaged site, an image taking condition, an imaged range, a reconstruction condition, operations of the gantry apparatus 10 (the imaging system 19), operations of the table apparatus 30, and/or the like.

Further, the memory 41 has stored therein an exclusive program for realizing a system controlling function 451, a pre-processing function 452, a reconstruction processing function 453, and an image processing function 454 (explained later).

The display 42 is a monitor referenced by the operator and is configured to display various types of information. For example, the display 42 is configured to output a medical image (a CT image) generated by the processing circuitry 45, a Graphical User Interface (GUI) used for receiving various types of operations from the operator, and the like. For example, the display 42 maybe a liquid crystal display or a Cathode Ray Tube (CRT) display.

The input interface 43 is configured to receive the various types of input operations from the operator, to convert the received input operations into electrical signals, and to output the electrical signals to the processing circuitry 45. For example, the input interface 43 is configured to receive, from the operator, an acquisition condition used at the time of acquiring the projection data, the reconstruction condition used at the time of reconstructing the CT image, an image processing condition used at the time of generating a post-processing image from the CT image, and/or the like.

Further, for example, the input interface 43 is realized by using a mouse, a keyboard, a trackball, a switch, a button, a joystick, and/or the like. Further, the input interface 43 may be provided for the gantry apparatus 10. Alternatively, the input interface 43 maybe configured by using a tablet terminal or the like capable of wirelessly communicating with the console apparatus 40 main body.

The processing circuitry 45 is configured to control operations of the entirety of the X-ray CT apparatus 1. The processing circuitry 45 includes, for example, the system controlling function 451, the pre-processing function 452, the reconstruction processing function 453, and the image processing function 454.

In the present embodiment, processing functions implemented by the constituent elements, namely, the system controlling function 451, the pre-processing function 452, the reconstruction processing function 453, and the image processing function 454, are stored in the memory 41 in the form of computer-executable programs. The processing circuitry 45 is a processor configured to realize the functions corresponding to the programs by reading and executing the programs from the memory 41.

In other words, the processing circuitry 45 that has read the programs has the functions illustrated within the processing circuitry 45 in FIG. 2.

Further, although the example was explained with reference to FIG. 2 in which the single piece of processing circuitry (the processing circuitry 45) realizes the processing functions implemented by the system controlling function 451, the pre-processing function 452, the reconstruction processing function 453, and the image processing function 454, it is also acceptable to structure the processing circuitry 45 by combining together a plurality of independent processors so that the functions are realized as a result of the processors executing the programs.

In other words, each of the abovementioned functions may be configured as a program so that the single piece of processing circuitry executes the programs. Alternatively, one or more specific functions may be installed in exclusive and independent program executing circuitry.

The system controlling function 451 is configured to control various types of functions of the processing circuitry 45, on the basis of the input operations received from the operator via the input interface 43. For example, via the input interface 43, the system controlling function 451 is configured to receive inputs of user information (e.g., a user ID) for a log-in, patient information, and/or the like. Further, for example, via the input interface 43, the system controlling function 451 is configured to receive an input of the imaging protocol.

The pre-processing function 452 is configured to generate data obtained by performing pre-processing processes such as a logarithmic conversion process, an offset process, an inter-channel sensitivity correction process, a beam hardening correction, and/or the like, on the detection data output from the DAS 18. The data (the detection data) prior to the pre-processing processes and the data resulting from the pre-processing processes may collectively be referred to as projection data.

The reconstruction processing function 453 is configured to generate CT image data, by performing, according to the reconstruction condition, a reconstructing process using a filtered back projection method, a successive approximation method, or the like, on the projection data generated by the pre-processing function 452. The generated CT image data is transmitted to the medical image processing apparatus 2, so that the medical image processing apparatus 2 performs a process of measuring an index for understanding hemodynamics.

On the basis of the input operations received from the operator via the input interface 43, the image processing function 454 is configured to convert the CT image data into tomographic image data on an arbitrary cross-sectional plane or three-dimensional image data, by using a publicly-known method. The three-dimensional image data is an example of the three-dimensional medical image data. Alternatively, the three-dimensional image data may be generated by the reconstruction processing function 453.

Further, post-processing processes may be performed by either the console apparatus 40 or the medical image processing apparatus 2. In another example, the post-processing processes may be performed by both the console apparatus 40 and the medical image processing apparatus 2 at the same time.

The post-processing processes defined in this situation are based on the concept denoting processes performed on the CT image data. For example, the post-processing processes are processes including a noise eliminating process, a Multi-Planar Reconstruction (MPR) display of a plurality of super-resolution slice images, a volume data rendering process, and/or the like.

With reference back to FIG. 1, the medical image processing apparatus 2 will be explained. The medical image processing apparatus 2 is an apparatus configured to measure the index for understanding hemodynamics of the heart, by using the three-dimensional image data generated from a scan performed on the patient P by the X-ray CT apparatus 1. In the present embodiment, an example will be explained in which, on the basis of three-dimensional image data from scanning a region including the heart, the medical image processing apparatus 2 is configured to measure an AMA indicating an angle formed by the mitral valve axis and the aortic valve axis. The AMA is used as an index indicating a stenosis state of the LVOT, for example.

For instance, as illustrated in FIG. 1, the medical image processing apparatus 2 includes a memory 21, a display 22, an input interface 23, and processing circuitry 24.

The memory 21 is configured to store therein various types of information. For example, the memory 21 has stored therein a program for enabling the circuitry included in the medical image processing apparatus 2 to realize functions thereof. Further, for example, the memory 21 has stored therein data received from the X-ray CT apparatus 1 and data generated by the processing circuitry 24.

In this situation, the memory 21 is realized by using a semiconductor memory element such as a Random Access Memory (RAM) or a flash memory, or a hard disk, an optical disc, or the like. Alternatively, the memory 21 maybe realized by using a server group (a cloud) connected to the medical image processing apparatus 2 via the network NW.

The display 22 is configured to display various types of information. For example, the display 22 is configured to display a Graphical User Interface (GUI) used for receiving various types of instructions, settings, and the like from a user via the input interface 23. For example, the display 22 maybe a liquid crystal display or a Cathode Ray Tube (CRT) display. The display 22 may be or a desktop type or may be configured by using a tablet terminal or the like capable of wirelessly communicating with the medical image processing apparatus 2 main body.

The input interface 23 is configured to receive various types of input operations from the user, to convert the received input operations into electrical signals, and to output the electrical signals to the processing circuitry 24. For example, the input interface 23 is realized by using a mouse, a keyboard, a trackball, a switch, a button, a joystick, a touchpad on which input operations can be performed by touching an operation surface thereof, a touch screen in which a display screen and a touchpad are integrally formed, contactless input circuitry using an optical sensor, audio input circuitry, and/or the like.

Alternatively, the input interface 23 may be configured by using a tablet terminal or the like capable of wirelessly communicating with the medical image processing apparatus 2 main body. Further, the input interface 23 may be circuitry configured to receive the input operations from the user through motion capture. In an example, the input interface 23 may be able to receive, as an input operation, a body motion, a line of sight, or the like of the user, by processing a signal obtained via a tracker or an image acquired of the user.

Further, the input interface 23 does not necessarily have to include physical operational component parts such as the mouse, the keyboard, and/or the like. For instance, possible examples of the input interface 23 include electrical signal processing circuitry configured to receive an electrical signal corresponding to an input operation from an external input mechanism provided separately from the medical image processing apparatus 2 and to output the electrical signal to the processing circuitry 24.

The processing circuitry 24 is configured to control operations of the entirety of the medical image processing apparatus 2, by implementing an obtaining function 241, a first specifying function 242, a second specifying function 243, a third specifying function 244, a fourth specifying function 245, a calculating function 246, and a display controlling function 247.

In the medical image processing apparatus 2 illustrated in FIG. 1, the processing functions are stored in the memory 21 in the form of computer-executable programs. The processing circuitry 24 is a processor configured to realize the functions corresponding to the programs, by reading and executing the programs from the memory 21. In other words, the processing circuitry 24 that has read the programs has the functions corresponding to the read programs.

Further, although the example was explained with reference to FIG. 1 in which the single piece of processing circuitry (the processing circuitry 24) realizes the obtaining function 241, the first specifying function 242, the second specifying function 243, the third specifying function 244, the fourth specifying function 245, the calculating function 246, and the display controlling function 247, it is also acceptable to structure the processing circuitry 24 by combining together a plurality of independent processors so that the functions are realized as a result of the processors executing the programs. Further, the processing functions included in the processing circuitry 24 maybe realized as being distributed among or incorporated into one or more pieces of processing circuitry, as appropriate.

Further, the processing circuitry 24 may be configured to realize the functions by using a processor of an external apparatus connected via the network NW. For example, the processing circuitry 24 may be configured to realize the functions illustrated in FIG. 1, by reading and executing the programs corresponding to the functions from the memory 21 and also using a server group (a cloud) connected to the medical image processing apparatus 2 via the network NW as computation resources.

The obtaining function 241 is configured to obtain the three-dimensional image data. For example, the obtaining function 241 is configured to receive the three-dimensional image data from the X-ray CT apparatus 1 via the network NW. Alternatively, the obtaining function 241 may obtain the three-dimensional image data from an external apparatus such as a PACS. In the present embodiment, the three-dimensional image data is three-dimensional image data obtained by scanning a site of the patient P including the heart. The three-dimensional image data does not necessarily need to render the entire heart of the patient P, as long as at least the left atrium, the mitral valve, the left ventricle, the aortic valves, and the aorta are rendered.

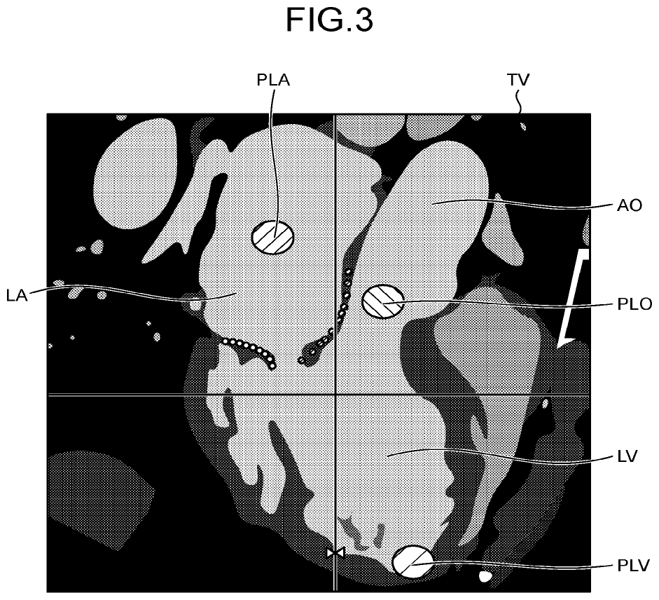

On the basis of the three-dimensional image data obtained by the obtaining function 241, the first specifying function 242 is configured to specify a left atrium region, a left ventricle region, and an aortic valve region. Further, the first specifying function 242 is configured to specify a three-chamber long-axis cross-sectional plane, on the basis of the left atrium region, the left ventricle region, and the aortic valve region. For example, the three-chamber long-axis cross-sectional plane is a cross-sectional view of the heart rendering the left atrium, the left ventricle, and the aorta. In the following sections, operations performed by the first specifying function 242 will be explained, with reference to FIG. 3. FIG. 3 is a drawing for explaining an example of the process of specifying the three-chamber long-axis cross-sectional plane.

To begin with, for example, the first specifying function 242 is configured to specify, from the three-dimensional image data, a region rendering the left atrium, the left ventricle, and the aortic valves, by using a publicly-known segmentation technique. In the following sections, a region rendering the left atrium may be referred to as a left atrium region; a region rendering the left ventricle may be referred to as a left ventricle region; and a region rendering the aortic valves may be referred to as an aortic valve region.

Subsequently, the first specifying function 242 is configured to specify a feature point PLA of the left atrium region. In an example, the first specifying function 242 is configured to specify center-of-gravity coordinates of the specified left atrium region, as the feature point PLA.

After that, the first specifying function 242 is configured to specify a feature point PLV of the left ventricle region. In an example, the first specifying function 242 is configured to calculate a first principal component by performing a principal component analysis on the coordinates of the left ventricle region. Subsequently, the first specifying function 242 is configured to project the left ventricle region coordinates onto an axis expressed by the first principal component and to specify, as the feature point PLV, the coordinates (the coordinates of the apex of the left ventricle) having the largest L2 norm value from the center-of-gravity coordinates of the left ventricle.

After that, the first specifying function 242 is configured to specify a feature point PLO of the aortic valve region. In an example, the first specifying function 242 is configured to specify, as the feature point PLO, center-of-gravity coordinates of three nadir points of the aortic valves. Further, the first specifying function 242 is configured to specify a cross-sectional plane that passes through the three feature points PLA, PLV, and PLO, as a three-chamber long-axis cross-sectional plane TV.

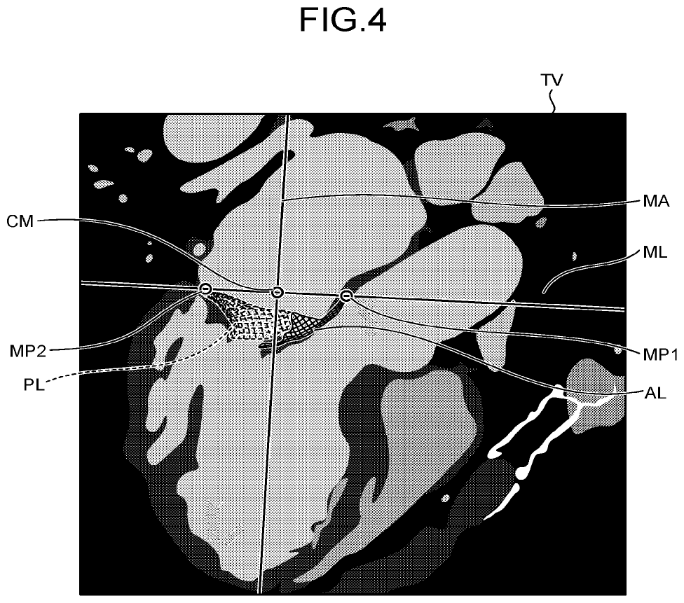

Explanations will continue with reference back to FIG. 1. The second specifying function 243 is configured to specify a mitral valve region from the three-chamber long-axis cross-sectional plane and to specify a mitral valve axis on the basis of a first annulus region included in the mitral valve region and the three-chamber long-axis cross-sectional plane. Next, operations performed by the second specifying function 243 will be explained, with reference to FIG. 4. FIG. 4 is a drawing for explaining an example of the process of specifying the mitral valve axis. FIG. 4 illustrates an example of a cross-sectional image rendering the three-chamber long-axis cross-sectional plane specified by the first specifying function 242.

To begin with, the second specifying function 243 is configured to specify an intersection point MP1 between the three-chamber long-axis cross-sectional plane TV and an annulus region of a mitral valve anterior leaflet AL. Subsequently, the second specifying function 243 is configured to specify an intersection point MP2 between the three-chamber long-axis cross-sectional plane TV and an annulus region of a mitral valve posterior leaflet PL. The annulus regions of the mitral valve anterior leaflet AL and the mitral valve posterior leaflet PL are examples of the first annulus region.

After that, the second specifying function 243 is configured to specify a middle point CM between the intersection point MP1 and the intersection point MP2. After that, the second specifying function 243 is configured to specify, as a mitral valve axis MA, a straight line that passes through the middle point CM, is orthogonal to a straight line ML connecting the intersection point MP1 to the intersection point MP2, and traverses across the three-chamber long-axis cross-sectional plane TV.

Explanations will continue with reference back to FIG. 1. The third specifying function 244 is configured to specify an anatomical structure in each of the valve regions from the three-chamber long-axis cross-sectional plane. For example, from the three-chamber long-axis cross-sectional plane specified by the first specifying function 242, the third specifying function 244 is configured to specify an anterior leaflet region included in the mitral valve region and a second annulus region included in the aortic valve region.

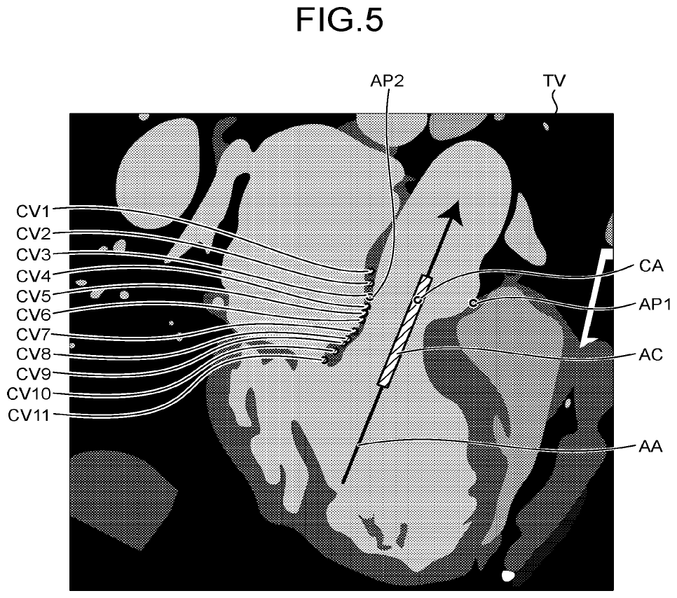

The fourth specifying function 245 is configured to specify an aortic valve axis. For example, the fourth specifying function 245 is configured to specify the aortic valve axis on the basis of a positional relationship between the anterior leaflet region and the second annulus region specified by the third specifying function 244 on the three-chamber long-axis cross-sectional plane. Next, operations performed by the third specifying function 244 and the fourth specifying function 245 will be explained, with reference to FIG. 5. FIG. 5 is a drawing for explaining an example of the process of specifying the aortic valve axis.

For example, the third specifying function 244 is configured to specify intersection points between the mitral valve anterior leaflet region and the three-chamber long-axis cross-sectional plane, on the basis of the mitral valve anterior leaflet region and the three-chamber long-axis cross-sectional plane in the three-dimensional image data. FIG. 5 illustrates an example in which points CV1 to CV11 are specified as the intersection points between the mitral valve anterior leaflet region and the three-chamber long-axis cross-sectional plane TV. Subsequently, the fourth specifying function 245 is configured to derive an approximate straight line AC of the intersection points CV1 to CV11. In the explanation below, the side on which the intersection point CV1 is positioned will be referred to as a basal part side, whereas the side on which the intersection point CV11 is positioned will be referred to as a tip end side.

Subsequently, the fourth specifying function 245 is configured to specify an intersection point AP1 between the three-chamber long-axis cross-sectional plane TV and an annulus region of the left semilunar valve (not illustrated) among the aortic valves. After that, the second specifying function 243 is configured to specify an intersection point AP2 between the three-chamber long-axis cross-sectional plane TV and an annulus region of the right semilunar valve (not illustrated) among the aortic valves. The annulus region of the left semilunar valve among the aortic valves and the annulus region of the right semilunar valve among the aortic valves are each an example of the second annulus region.

Alternatively, in place of the annulus region of the left semilunar valve among the aortic valves and the annulus region of the right semilunar valve among the aortic valves, the second specifying function 243 may be configured to specify the three-chamber long-axis cross-sectional plane TV and an annulus region of the posterior semilunar valve among the aortic valves, as the second annulus region.

Subsequently, the fourth specifying function 245 is configured to specify a middle point CA between the intersection point AP1 and the intersection point AP2. Further, the fourth specifying function 245 is configured to specify a straight line that passes through the middle point CA, extends parallel to the approximate straight line AC, and traverses across the three-chamber long-axis cross-sectional plane TV, as an aortic valve axis AA.

As explained above, the aortic valve axis in the present embodiment is specified on the basis of the shape of the mitral valve anterior leaflet. For example, when the LVOT has a stenosis due to hypertrophic cardiomyopathy or the like, the mitral valve anterior leaflet exhibits a shape that is pulled toward the side where the aortic valves are positioned. As a result, the aortic valve axis based on the shape of the mitral valve anterior leaflet reflects the state of the LVOT (e.g., a degree of the stenosis).

In the example in FIG. 5, the fourth specifying function 245 specifies the approximate straight line from all of the specified intersection points (the intersection points CV1 to CV11). However, the fourth specifying function 245 may be configured to specify an approximate straight line of a plurality of intersection points positioned on the basal part side (e.g., CV1 to CV5 in the example in FIG. 5).

The reason is that the tip end part of the mitral valve may have a bent shape in some situations due to movements of the mitral valve. In those situations, if the intersection points positioned on the tip end side were used for specifying an approximate straight line, there would be a possibility that an aortic valve axis to be specified might not properly reflect the state of the LVOT, because of an impact from the movements of the mitral valve. In other words, by specifying the approximate straight line while using the intersection points positioned on the basal part side, the fourth specifying function 245 is able to specify the aortic valve axis reflecting the state of the LVOT.

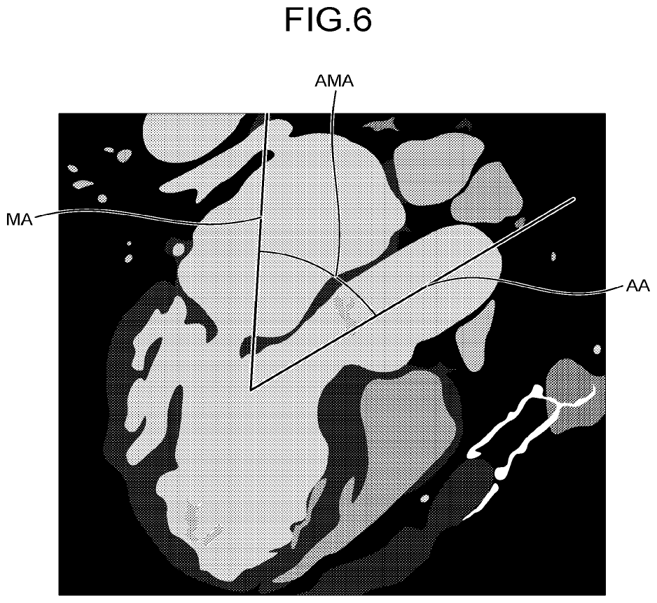

Explanations will continue with reference back to FIG. 1. The calculating function 246 is configured to calculate an angle based on the mitral valve axis and the aortic valve axis. Next, operations performed by the calculating function 246 will be explained, with reference to FIG. 6. FIG. 6 is a drawing for explaining an example of a process of calculating the AMA. As illustrated in FIG. 6, as the AMA, the calculating function 246 is configured to calculate the angle formed by the mitral valve axis MA specified by the second specifying function 243 and the aortic valve axis AA specified by the fourth specifying function 245.

The AMA is the angle derived by using the aortic valve axis AA in FIG. 5. As explained above, the aortic valve axis AA reflects the state of the LVOT. Consequently, it can be said that the AMA calculated by the calculating function 246 is a proper index indicating the state of the LVOT.

Explanations will continue with reference back to FIG. 1. The display controlling function 247 is configured to exercise control so that various types of information are displayed. For example, the display controlling function 247 is configured to exercise control so that the display 22 displays a display screen displaying the AMA calculated by the calculating function 246.

Alternatively, the display controlling function 247 may cause the display 22 to display a screen related to the deriving of the AMA (i.e., a screen displaying FIGS. 4 to 6 explained above).

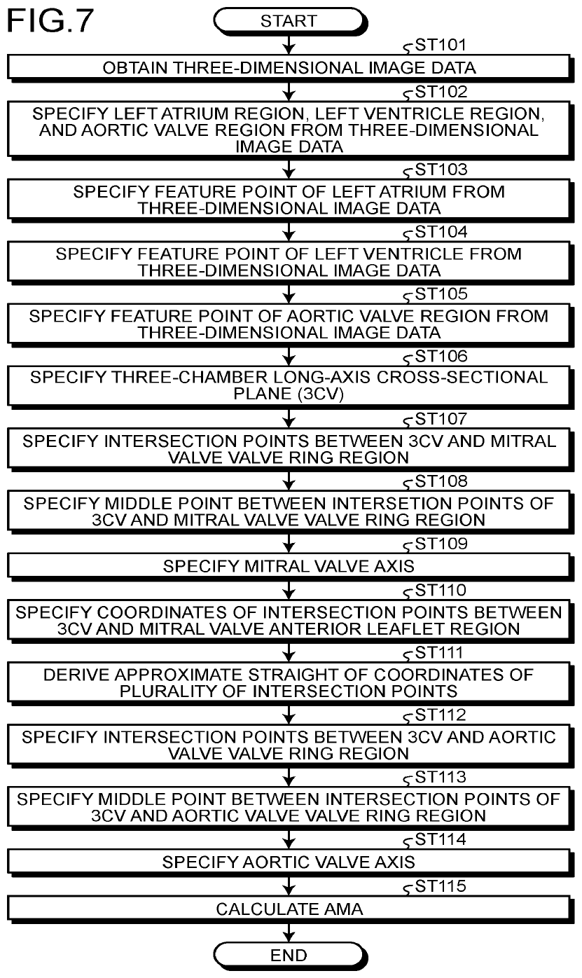

Next, processes performed by the medical image processing apparatus 2 will be explained. FIG. 7 is a flowchart illustrating an example of the processes performed by the medical image processing apparatus 2.

To begin with, the obtaining function 241 obtains three-dimensional image data (step ST101). For example, from the X-ray CT apparatus 1, the obtaining function 241 receives three-dimensional image data rendering the heart of the patient P, via the network NW.

Subsequently, the first specifying function 242 specifies a left atrium region, a left ventricle region, and an aortic valve region, from the three-dimensional image data (step ST102). For example, by using a publicly-known segmentation technique, the first specifying function 242 specifies the left atrium region, the left ventricle region, and the aortic valve region from the three-dimensional image data.

After that, the first specifying function 242 specifies a feature point of the left atrium region from the three-dimensional image data (step ST103). For example, the first specifying function 242 calculates center-of-gravity coordinates of the left atrium region and specifies the center-of-gravity coordinates as the feature point of the left atrium region.

After that, the first specifying function 242 specifies a feature point of the left ventricle region from the three-dimensional image data (step ST104). For example, the first specifying function 242 performs a principal component analysis on the left ventricle region coordinates, calculates the coordinates of the apex of the left ventricle on the basis of a result of the analysis, and specifies the apex coordinates as the feature point of the left ventricle region.

Subsequently, the first specifying function 242 specifies a feature point of the aortic valve region from the three-dimensional image data (step ST105). For example, the first specifying function 242 specifies center-of-gravity coordinates of three nadir points of the aortic valves as the feature point of the aortic valve region.

After that, the first specifying function 242 specifies a three-chamber long-axis cross-sectional plane from the three-dimensional image data (step ST106). For example, as the three-chamber long-axis cross-sectional plane, the first specifying function 242 specifies a cross-sectional plane (3CV) that traverses through the feature point of the left atrium region, the feature point of the left ventricle region, and the feature point of the aortic valve region specified at step ST103 through ST105.

Subsequently, the second specifying function 243 specifies intersection points between the three-chamber long-axis cross-sectional plane and the mitral valve annulus region (step ST107). For example, by using a publicly-known segmentation technique, the second specifying function 243 specifies an annulus region of the mitral valve anterior leaflet and an annulus region of the mitral valve posterior leaflet, from the three-dimensional image data. After that, the second specifying function 243 specifies an intersection point between the three-chamber long-axis cross-sectional plane and the annulus region of the mitral valve anterior leaflet and an intersection point between the three-chamber long-axis cross-sectional plane and the annulus region of the mitral valve posterior leaflet.

Subsequently, the second specifying function 243 specifies the middle point between the intersection points of the three-chamber long-axis cross-sectional plane and the mitral valve annulus region (step ST108). For example, the second specifying function 243 calculates the distance between the two intersection points specified at step ST107 and further specifies the middle point between the two intersection points.

After that, the second specifying function 243 specifies a mitral valve axis (step ST109). For example, as the mitral valve axis, the second specifying function 243 specifies a straight line that is perpendicular to the straight line connecting together the two intersection points specified at step ST107, passes through the middle point specified at step ST108, and traverses across the three-chamber long-axis cross-sectional plane.

Subsequently, the third specifying function 244 specifies the coordinates of intersection points between the three-chamber long-axis cross-sectional plane and the mitral valve anterior leaflet region (step ST110). For example, by using a publicly-known segmentation technique, the third specifying function 244 specifies the plurality of intersection points between the three-chamber long-axis cross-sectional plane and the mitral valve anterior leaflet region, from the three-chamber long-axis cross-sectional plane. After that, the fourth specifying function 245 specifies an approximate straight line of the plurality of intersection points specified at step ST110 (step ST111).

Subsequently, the fourth specifying function 245 specifies intersection points between the three-chamber long-axis cross-sectional plane and the aortic valve annulus region (step ST112). For example, by using a publicly-known segmentation technique, the third specifying function 244 specifies the annulus region of the aortic valves from the three-dimensional image data. After that, the fourth specifying function 245 specifies an intersection point between the three-chamber long-axis cross-sectional plane and an annulus region of the left semilunar valve among the aortic valves and an intersection point between the three-chamber long-axis cross-sectional plane and an annulus region of the right semilunar valve among the aortic valves.

Subsequently, the fourth specifying function 245 specifies the middle point between the intersection points of the three-chamber long-axis cross-sectional plane and the aortic annulus region (step ST113). For example, the fourth specifying function 245 calculates the distance between the two intersection points specified at step ST112 and specifies the middle point between the two intersection points.

After that, the fourth specifying function 245 specifies an aortic valve axis (step ST114). For example, as the aortic valve axis, the fourth specifying function 245 specifies a straight line that extends parallel to the approximate straight line specified at step ST111, passes through the middle point specified at step ST113, and traverses across the three-chamber long-axis cross-sectional plane.

Subsequently, the calculating function 246 calculates the angle formed by the mitral valve axis specified at step ST109 and the aortic valve axis specified at step ST114 as an AMA (step ST115). The present processes are thus ended.

The medical image processing apparatus 2 according to the embodiment described above is configured: to specify the three-chamber long-axis cross-sectional plane from the three-dimensional image data; to specify, from the three-chamber long-axis cross-sectional plane, the mitral valve axis on the basis of the annulus region of the mitral valve; to specify the anterior leaflet region of the mitral valve from the three-chamber long-axis cross-sectional plane; to specify the aortic valve axis on the basis of the annulus regions of the aortic valves on the three-chamber long-axis cross-sectional plane, the three-chamber long-axis cross-sectional plane, and the anterior leaflet region; and to calculate the angle based on the mitral valve axis and the aortic valve axis.

With this configuration, the medical image processing apparatus 2 according to the present embodiment is able to specify, from the three-chamber long-axis cross-sectional plane, the aortic valve axis reflecting the shape of the mitral valve anterior leaflet. Because the shape of the mitral valve anterior leaflet changes in accordance with states of the LVOT (e.g., degrees of a stenosis), the medical image processing apparatus 2 according to the present embodiment is able to measure the AMA reflecting the state of the LVOT.

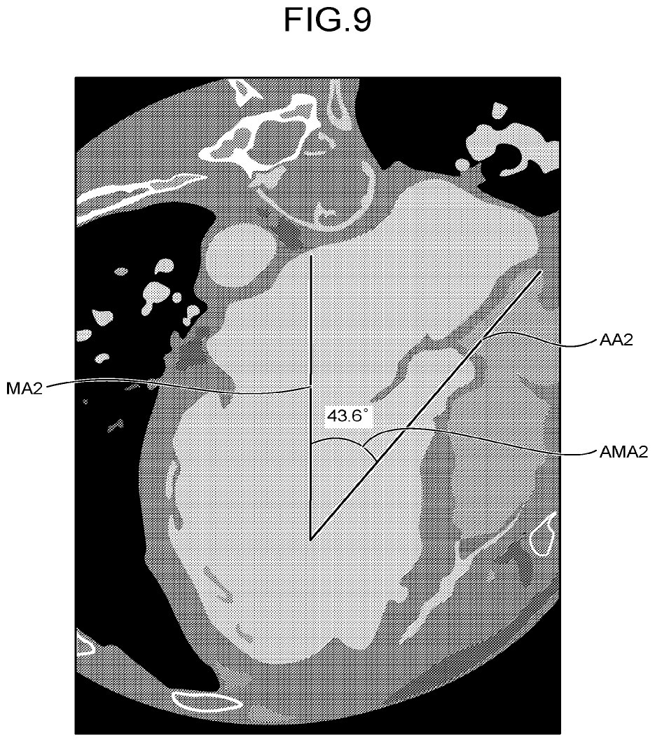

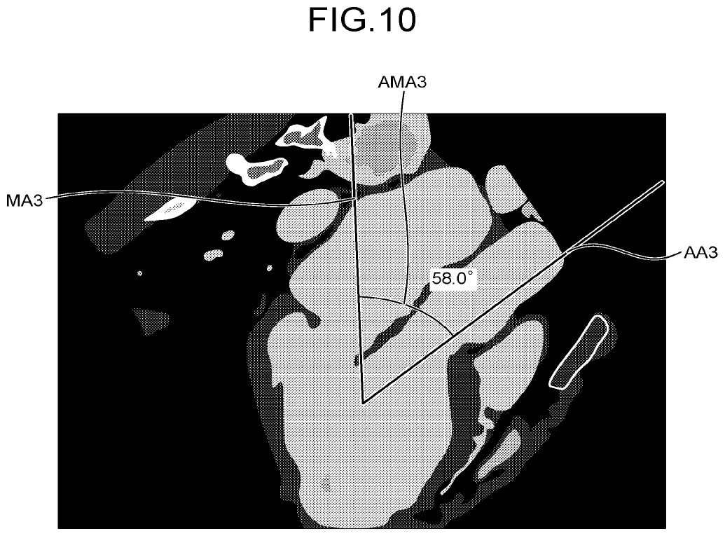

Next, to make a comparison with the processes of calculating (measuring) the AMA in the present embodiment illustrated in FIGS. 3 to 6, examples of results of measuring AMAs using processes different from those in the present embodiment are illustrated in FIGS. 9 and 10. FIG. 9 is a drawing for explaining an example of an AMA measuring result from processes different from those in the embodiment.

FIG. 9 illustrates the example in which a medical doctor has visually set a mitral valve axis MA2 and an aortic valve axis AA2 on a three-chamber long-axis cross-sectional plane and measured AMA2, which is the angle formed by the mitral valve axis MA2 and the aortic valve axis AA2. In the example in FIG. 9, because the medical doctor visually set the mitral valve axis MA2 and the aortic valve axis AA2, it can be said that the aortic valve axis AA2 being set expresses a blood flow aimed by the medical doctor. In the example in FIG. 9, AMA2 is equal to 43.6°.

Further, FIG. 10 is a drawing for explaining an example of another AMA calculation result obtained by a medical image processing apparatus configured differently from the embodiment. FIG. 10 illustrates the example in which the medical image processing apparatus configured differently from the present embodiment has set a mitral valve axis MA3 and an aortic valve axis AA3, on the same three-chamber long-axis cross-sectional plane of the same patient P as those in FIG. 9 and measured AMA3, which is the angle formed by the mitral valve axis MA3 and the aortic valve axis AA3.

In the example in FIG. 10, processing circuitry (hereinafter, “the other processing circuitry”) of the other medical image processing apparatus is configured to specify, from the three-chamber long-axis cross-sectional plane, a straight line connecting together points on the mitral valve annulus from the three-chamber long-axis cross-sectional plane and to specify a line perpendicular to the straight line as the mitral valve axis MA3.

Further, the other processing circuitry is configured to specify, from the three-chamber long-axis cross-sectional plane, a straight line connecting together points on the aortic valve annulus from the three-chamber long-axis cross-sectional plane and to specify a line perpendicular to the straight line as the aortic valve axis AA3. Further, the other processing circuitry is configured to measure AMA3, which is the angle formed by the mitral valve axis MA3 and the aortic valve axis AA3. In the example in FIG. 10, AMA3 is equal to 58.0°.

The absolute value of the difference between AMA2 in FIG. 9 and AMA3 in FIG. 10 is 14.2°. It is considered that AMA3 in FIG. 10 cannot be regarded as expressing a blood flow aimed by the medical doctor. In this situation, because it is considered that the medical doctor imagines a blood flow while taking the state of the LVOT into consideration, it can be said that the aortic valve axis AA2 in FIG. 9 is reflecting the state of the LVOT. In contrast, the aortic valve axis AA3 in FIG. 10 is set without using the shape of the mitral valve anterior leaflet. In other words, there is a possibility that the aortic valve axis AA3 in FIG. 10 may be unable to reflect the state of the LVOT.

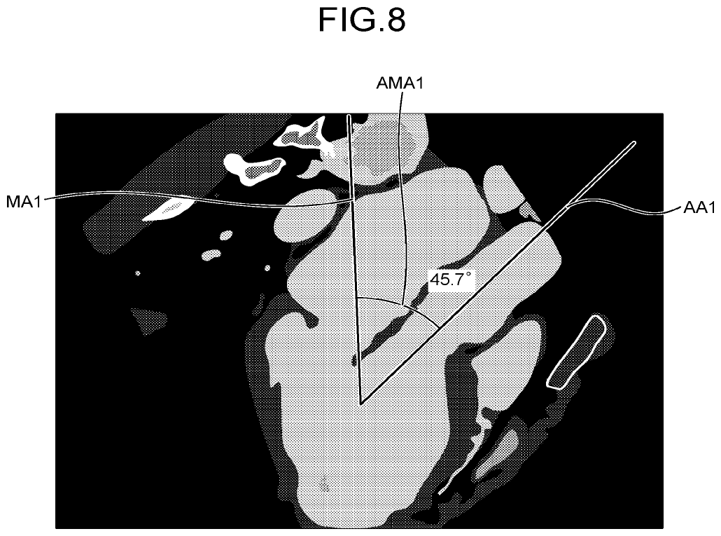

In comparison, as explained above, the medical image processing apparatus 2 according to the present embodiment is capable of specifying the aortic valve axis reflecting the state of the LVOT. In this situation, FIG. 8 is a drawing for explaining an example of the AMA calculation result obtained by the medical image processing apparatus 2 according to the embodiment.

FIG. 8 illustrates the example in which the medical image processing apparatus 2 according to the present embodiment has set a mitral valve axis MA1 and an aortic valve axis AA1 on the same three-chamber long-axis cross-sectional plane of the same patient P as those in FIG. 9 and measured AMA1, which is the angle formed by the mitral valve axis MA1 and the aortic valve axis AA1.

In the example in FIG. 8, AMA1 is equal to 45.7°. The absolute value of the difference between AMA2 in FIG. 9 and AMA1 in FIG. 8 is 1.9° and is smaller than the absolute value of the difference between AMA2 in FIG. 9 and AMA3 in FIG. 10. Accordingly, it can be said that the aortic valve axis AA1 in FIG. 8 better reflects the state of the LVOT than the aortic valve axis AA3 in FIG. 10. As explained herein, the medical image processing apparatus 2 according to the present embodiment is able to accurately and efficiently measure the index for understanding hemodynamics of the heart.

Further, it is possible to carry out any of the embodiments described above with an appropriate modification, by changing a part of the configurations or the functions of the apparatuses. Thus, in the following sections, a number of modification examples of the embodiments described above will be explained as other embodiments. In the following sections, differences from the above embodiments will primarily be explained, and detailed explanations of some of the features that are the same as those previously explained will be omitted. Further, the modification examples described below may be carried out individually or may be carried out in combination as appropriate.

### FIRST MODIFICATION EXAMPLE

In the above embodiment, the example was explained in which the medical image diagnosis apparatus 1 is an X-ray CT apparatus; however, possible embodiments are not limited to this example. For instance, the medical image diagnosis apparatus 1 maybe a Magnetic Resonance Imaging (MRI) apparatus, an Angio-CT system, a tomosynthesis apparatus, a Single Photon Emission Computed Tomography (SPECT) apparatus, a Positron Emission computed Tomography (PET) apparatus, an ultrasound image diagnosis apparatus, or the like.

According to the present modification example, it is possible to accurately and efficiently measure the index for understanding hemodynamics of the heart by using three-dimensional medical image data obtained by not only an X-ray CT apparatus, but also other various medical image diagnosis apparatuses.

### SECOND MODIFICATION EXAMPLE

In the above embodiment, the example was explained in which the information processing apparatus separate from the console apparatus 40 of the X-ray CT apparatus 1 serves as the medical image processing apparatus 2; however, the console apparatus 40 maybe provided with the function configuration included in the medical image processing apparatus 2. According to the present modification example, it is possible to accurately and efficiently measure the index for understanding hemodynamics of the heart by using the X-ray CT apparatus 1 alone.

### THIRD MODIFICATION EXAMPLE

In the above embodiment, the example was explained in which no equipment is placed with the heart of the patient P; however, the above embodiment is also applicable to situations where equipment is placed with the heart of the patient P. In the following sections, an example will be explained in which a piece of mitral valve treatment equipment is placed with the mitral valve of the patient P.

In this situation, on the basis of the shape of the mitral valve and the shape of the mitral valve treatment equipment, the second specifying function 243 according to the present modification example is configured to specify, from three-dimensional image data, a region expected to have the mitral valve if no mitral valve treatment equipment were placed for the patient P, as a mitral valve region. Further, similarly, the second specifying function 243 is configured to specify a region expected to have the annulus of the mitral valve if no mitral valve treatment equipment were placed, as a mitral valve annulus region.

The medical image processing apparatus 2 according to the present modification example is able to specify an aortic valve axis reflecting the state of the LVOT, even in the situation where the shapes of the left ventricle and/or the left ventricular outflow tract are deformed by the mitral valve treatment equipment, for example.

### FOURTH MODIFICATION EXAMPLE

In the above embodiment, the example was explained in which the processes of calculating the index for understanding hemodynamics of the heart were performed on the left heart system (the left atrium, the mitral valve, the left ventricle, the aortic valves, and the aorta); however, the above embodiment is also applicable to the situation where the processes are performed on a right heart system (the right atrium, the tricuspid valve, the right ventricle, the pulmonary artery valves, and the pulmonary artery).

When the processes are performed on the right heart system, it is possible to apply the processes of the above embodiment by replacing, in the above embodiment, the “left atrium” with the “right atrium”, the “mitral valve” with the “tricuspid valve”, the “left ventricle” with the “right ventricle”, the “Left Ventricular Outflow Tract (LVOT)” with the “Right Ventricular Outflow Tract (RVOT)”, the “aorta” with the “pulmonary artery”, and the “three-chamber long-axis cross-sectional plane” with a “long-axis cross-sectional plane that traverses across a right atrium region, a right ventricle region, and a pulmonary artery valve region”.

According to the present modification example, it is possible to accurately and efficiently measure the index for understanding hemodynamics of the heart, with regard to not only the left heart system, but also the right heart system.

According to at least one aspect of the embodiments, the modification examples, and the like described above, it is possible to accurately and efficiently measure the index for understanding hemodynamics of the heart.

The term “processor” used in the above description denotes, for example, a Central Processing Unit (CPU), a Graphical Processing Unit (GPU), or circuitry such as an Application Specific Integrated Circuit (ASIC) or a programmable logic device (e.g., a Simple Programmable Logic Device (SPLD), a Complex Programmable Logic Device (CPLD), or a Field Programmable Gate Array (FPGA)).

One or more processors are configured to realize the functions by reading and executing the programs saved in the memory 41. Alternatively, instead of having the programs saved in the memory 41, it is also acceptable to directly incorporate the programs into the circuitry of the one or more processors. In that situation, the one or more processors are configured to realize the functions by reading and executing the programs incorporated in the circuitry thereof.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical image processing apparatus comprising processing circuitry configured:
to obtain three-dimensional medical image data rendering a heart;
to specify, on a basis of a left atrium region, a left ventricle region, and an aortic valve region rendered in the three-dimensional medical image data, a three-chamber long-axis cross-sectional plane that traverses across these regions;
to specify a mitral valve axis on a basis of a first annulus region included in a mitral valve region rendered in the three-dimensional medical image data and the three-chamber long-axis cross-sectional plane;
to specify, from the three-chamber long-axis cross-sectional plane, an anterior leaflet region included in the mitral valve region and a second annulus region included in the aortic valve region;
to specify an aortic valve axis on a basis of a positional relationship between the anterior leaflet region and the second annulus region on the three-chamber long-axis cross-sectional plane; and
to calculate an angle based on the mitral valve axis and the aortic valve axis.

2. The medical image processing apparatus according to claim 1, wherein the processing circuitry is configured to specify the three-chamber long-axis cross-sectional plane, on a basis of center-of-gravity coordinates of the left atrium region, coordinates of a tip end of the left ventricle region, and center-of-gravity coordinates of the aortic valve region.

3. The medical image processing apparatus according to claim 1, wherein the processing circuitry is configured:
to derive a straight line that passes through two intersection points at which the three-chamber long-axis cross-sectional plane intersects the first annulus region; and
to specify a straight line that is orthogonal to the straight line and passes through a middle point between sets of coordinates of the two intersection points, as the mitral valve axis.

4. The medical image processing apparatus according to claim 1, wherein the processing circuitry is configured:
to derive a middle point between two intersection points at which the second annulus region intersects the three-chamber long-axis cross-sectional plane and an approximate straight line based on coordinates of a plurality of intersection points between the three-chamber long-axis cross-sectional plane and the anterior leaflet region; and
to specify a straight line that passes through the middle point and extends parallel to the approximate straight line as the aortic valve axis.

5. The medical image processing apparatus according to claim 4, wherein the processing circuitry is configured to derive the approximate straight line that is based on two or more sets of coordinates positioned closer to a basal part side of the anterior leaflet region, among the coordinates of the plurality of intersection points.

6. The medical image processing apparatus according to claim 1, wherein the processing circuitry is configured to specify the mitral valve axis, on the basis of the first annulus region included in the mitral valve region rendered in the three-dimensional medical image data and the three-chamber long-axis cross-sectional plane, the mitral valve region corresponding to a situation where equipment is placed on a mitral valve and including a region where the mitral valve was present prior to the placement of the equipment.

7. A medical image processing method implemented by a medical image processing apparatus and comprising:
obtaining three-dimensional medical image data rendering a heart;
specifying, on a basis of a left atrium region, a left ventricle region, and an aortic valve region rendered in the three-dimensional medical image data, a three-chamber long-axis cross-sectional plane that traverses across these regions;
specifying a mitral valve axis on a basis of a first annulus region included in a mitral valve region rendered in the three-dimensional medical image data and the three-chamber long-axis cross-sectional plane;
specifying, from the three-chamber long-axis cross-sectional plane, an anterior leaflet region included in the mitral valve region and a second annulus region included in the aortic valve region;
specifying an aortic valve axis on a basis of a positional relationship between the anterior leaflet region and the second annulus region on the three-chamber long-axis cross-sectional plane; and
calculating an angle based on the mitral valve axis and the aortic valve axis.

8. A storage medium storing therein a program for:
obtaining three-dimensional medical image data rendering a heart;
specifying, on a basis of a left atrium region, a left ventricle region, and an aortic valve region rendered in the three-dimensional medical image data, a three-chamber long-axis cross-sectional plane that traverses across these regions;
specifying a mitral valve axis on a basis of a first annulus region included in a mitral valve region rendered in the three-dimensional medical image data and the three-chamber long-axis cross-sectional plane;
specifying, from the three-chamber long-axis cross-sectional plane, an anterior leaflet region included in the mitral valve region and a second annulus region included in the aortic valve region;
specifying an aortic valve axis on a basis of a positional relationship between the anterior leaflet region and the second annulus region on the three-chamber long-axis cross-sectional plane; and
calculating an angle based on the mitral valve axis and the aortic valve axis.

9. A medical image processing apparatus comprising processing circuitry configured:
to obtain three-dimensional medical image data rendering an atrium, a ventricle, a first valve positioned between the atrium and the ventricle, and a second valve positioned between the ventricle and an artery;
to specify, on a basis of an atrium region, a ventricle region, and a second valve region rendered in the three-dimensional medical image data, a long-axis cross-sectional plane that traverses across these regions;
to specify a first valve axis on a basis of a first annulus region included in a first valve region rendered in the three-dimensional medical image data and the long-axis cross-sectional plane;
to specify, from the long-axis cross-sectional plane, an anterior leaflet region included in the first valve region and a second annulus region included in the second valve region;
to specify a second valve axis on a basis of a positional relationship between the anterior leaflet region and the second annulus region on the long-axis cross-sectional plane; and
to calculate an angle based on the first valve axis and the second valve axis.
